(19) United States
(12) Patent Application Publication
(10) Pub. No.: US 2015/0031137 A1
(43) Pub. Date: Jan. 29, 2015

(54) WASTE WATER DISCHARGE APPARATUS AND PROCESS

(71) Applicant: Gregory Scott BOWERS, JR., (US)

(72) Inventor: Gregory Scott Bowers, JR., Greenville, SC (US)

(21) Appl. No.: 14/384,699

(22) PCT Filed: Mar. 13, 2013
(86) PCT No.: PCT/US2013/030843

§ 371 Date: Sep. 12, 2014

Related U.S. Application Data
(60) Provisional application No. 61/610,194, filed on Mar. 13, 2012.

(51) Int. Cl. G01N 33/18

(52) U.S. Cl. CPC G01N 33/18
USPC 436/73

(57) ABSTRACT

A process of monitoring a waste stream for metal levels and metal precipitating agent levels is provided.

### CROSS REFERENCE TO RELATED APPLICATION

This application claims the benefit of U.S. application Ser. No. 61/610,194, filed Mar. 13, 2012 and PCT/US213/030843 filed Mar. 13, 2013 both entitled “Waste Water Discharge Apparatus and Process” and whose entire contents are incorporated herein by reference in their entirety and for all purposes.

### FIELD OF THE INVENTION

This invention is directed towards an apparatus and process for monitoring treated waste water prior to discharge.

### BACKGROUND OF THE INVENTION

Industrial wastewaters commonly include a variety of contaminants which require treatment (i.e., removal) even before the wastewater can be discharged from the plant site. The nature of the wastewater contaminants is in large part dependent on the commercial processes practiced in the plant. Accordingly, there is great variety in the nature of wastewater contaminant problems. Moreover, the matrix (i.e., makeup) of wastewater even at a given commercial site will usually vary, sometimes dramatically, with changes in production or the like.

Particular industries, for example such as those relating to metal plating, metal finishing, or circuit board manufacturing activities, generate wastewater with heavy metals (e.g., copper, nickel, etc.) and other metals in solution with such wastewater. The commercial activities themselves may inherently generate heavy metals which are chelated and/or complexed for purposes of the commercial activity (e.g., metal plating) itself. Chelating and/or complexing tends to cause such metals to remain in solution, and thus require special attention for their removal.

During the typical course of plant activity, heavy metal concentration in the wastewater is highly variable. While concentration variations can in general be expected, monitoring of and reacting to specific variations is problematic. Concentrations of heavy metals may typically vary from a few parts per million to several hundred parts per million, even in a very short time, such as a matter of minutes.

Not only do concentration levels vary drastically, but also extreme variations can be experienced with respect to the matrix (both in identity and nature, e.g., chelated versus non-chelated) of heavy metals present.

In general, it is known to add (i.e., feed) various precipitating agents to wastewater to precipitate such heavy metals for their removal from the water. The amount of such precipitating agents required (i.e. consumed) in the course of precipitating such heavy metals of course depends on the degree of presence of such heavy metals in solution with the wastewater. Since effective real time monitoring of heavy metal concentration levels has heretofore generally proven difficult, such treatment chemical feeding (i.e. the feed rate of precipitating agents) is typically set at a compromise level, such as for precipitating the maximum expected concentration of heavy metals. Such a compromise setting creates an excess amount of sludge, which sludge may often be classified as a hazardous waste. Moreover, since the cost of the treatment chemicals is not insignificant, wasteful overfeeding thereof is costly.

Operators have been known to attempt periodic checks to manually detect the level of metals entering the wastewater (i.e., assess the expected concentrations), and adjust the chemical feed rate accordingly. However, such a manual adjustment merely alters the set feed rate in accordance with periodic reassessments of the anticipated maximum concentration, and does nothing to eliminate excess sludge production and excessive and costly chemical usage caused by differences between actual concentration levels and the anticipated maximums thereof. Moreover, short-term spikes can still occur, meaning that inadequately treated wastewater can be nonetheless discharged. Such occurrences are particularly problematic where applicable laws regulate the permissible discharge concentration levels, such as to certain fractional parts per million or certain parts per million.

In some industrial settings, anticipation of heavy metal concentrations in the wastewater may be relatively less “predictable”. For example, a totally unexpected occurrence of heavy metals in the wastewater can go unchecked, thereby causing the plant to exceed permissible discharge levels. For example, maintenance personnel might empty mop buckets or the like containing chelated heavy metals picked up from the floor of the facility, which could cause a heavy metal concentration spike in the wastewater at a time whenever commercial activity in the plant is nil, and precipitating agent feed pumps may be switched off. The plant is nonetheless responsible for its wastewater discharge, though no effective continuous monitoring systems for preventing such undesirable discharges may be available.

It is generally known that certain metals in solution in wastewater may be precipitated therefrom by controlling the pH level of the wastewater. For example, non-chelated and non-complexed metals in particular may be in various degrees precipitated in such manner. Automatic controllers are generally available which function to probe the wastewater for its pH level, and automatically pump treatment chemicals accordingly to the wastewater so as to adjust its pH level within an established deviation from a pre-selected set point. One example of such a controller is the Model 5 proportional pH pump controller, made by Chem-Tech International, Inc., of 92 Bolt Street, Lowell, Mass., 01853. While such a controller may be effective for metals which may be precipitated through such pH inducement, heavy metals which are chelated and/or complexed generally will not be precipitated with such pH level control. Thus, the monitoring and treatment problems noted above persist, and may be compounded where a changing mix of chelated and non-chelated metals is presented for treatment.

Another aspect of wastewater treatment problems where both such types of metals are in solution (i.e., which can and can not be practically precipitated through pH inducement) is that use of a precipitating agent can precipitate both such types of metals. However, unnecessary sludge production is caused by precipitating metals in such a manner which could have otherwise been precipitated through pH level control (as generally discussed above). Again, the amount of precipitating agent consumption is also a factor.

In addition to the availability of known pH level control generally outlined above, at least one other generally known method, involving a so-called oxidation reduction potential probe, attempts to address precipitating agent usage. Such a probe is typically used to detect the presence of excess (i.e. un-consumed) precipitating agent at a phase of a wastewater treatment program after all the metal is removed. One particular limitation of such a system is that it cannot distinguish between, for example, chelated and non-chelated metals, and must therefore feed precipitating agent until there is an excess of such agent present in the water. Feed control feedback also is derived from detected excess agent, not from information relative remaining metal in solution to be precipitated. Thus, there is no effective prevention of excess sludge generation or wasteful chemical usage.

Another limitation of a waste treatment system utilizing an oxidation reduction potential (ORP) probe is that the probe operation involves an electrical measurement which is affected by changes in the pH level of the wastewater, the amount of total dissolved solids therein, the oxidizer concentration, and the amount of chelated metal in the wastewater. Thus, an ORP probe system is inherently ineffective for use in providing close control of the feeding of chemical treatment solutions into wastewater treatment systems.

For example, U.S. Pat. Nos. 5,045,213; 4,999,116; and 4,923,599, all of are directed to wastewater treatment for the removal of heavy metals, which is optimized by continuously removing and filtering a sample flow of treated wastewater subject to pH level control to determine the presence of remaining metals in solution to be precipitated.

Thus, chemical precipitation is the most common technology used to remove dissolved (ionic) metals from solutions, such as process wastewaters containing toxic metals. The ionic metals are converted to an insoluble form (particle) by the chemical reaction between the soluble metal compounds and the precipitating reagent. The particles formed by this reaction are removed from solution by settling and/or filtration. The effectiveness of a chemical precipitation process is dependent on several factors, including the type and concentration of ionic metals present in solution, the precipitant used, the reaction conditions (especially the pH of the solution), and the presence of other constituents that may inhibit the precipitation reaction.

Further, in wastewater treatment operations, the processes of coagulation and flocculation are employed to separate suspended solids from water. Although the terms coagulation and flocculation are often used interchangeably, or the single term “flocculation” is used to describe both; they are, in fact, two distinct processes.

Finely dispersed solids (colloids) suspended in wastewaters are stabilized by negative electric charges on their surfaces, causing them to repel each other. Since this prevents these charged particles from colliding to form larger masses, called flocs, they do not settle. To assist in the removal of colloidal particles from suspension, chemical coagulation and flocculation are required. These processes, usually done in sequence, are a combination of physical and chemical procedures. Chemicals are mixed with wastewater to promote the aggregation of the suspended solids into particles large enough to settle or be removed.

Coagulation is the destabilization of colloids by neutralizing the forces that keep them apart. Cationic coagulants provide positive electric charges to reduce the negative charge (zeta potential) of the colloids. As a result, the particles collide to form larger particles (flocs). Rapid mixing is required to disperse the coagulant throughout the liquid. Care must be taken not to overdose the coagulants as this can cause a complete charge reversal and restabilize the colloid complex.

Flocculation is the action of polymers to form bridges between the flocs and bind the particles into large agglomerates or clumps. Bridging occurs when segments of the polymer chain adsorb on different particles and help particles aggregate. An anionic flocculant will react against a positively charged suspension, adsorbing on the particles and causing destabilization either by bridging or charge neutralization. In this process it is essential that the flocculating agent be added by slow and gentle mixing to allow for contact between the small flocs and to agglomerate them into larger particles. The newly formed agglomerated particles are quite fragile and can be broken apart by shear forces during mixing. Care must also be taken to not overdose the polymer as doing so will cause settling/clarification problems. Anionic polymers themselves are lighter than water. As a result, increasing the dosage will increase the tendency of the floc to float and not settle.

Once suspended particles are flocculated into larger particles, they can usually be removed from the liquid by sedimentation, provided that a sufficient density difference exists between the suspended matter and the liquid. Such particles can also be removed or separated by media filtration, straining or floatation. When a filtering process is used, the addition of a flocculant may not be required since the particles formed by the coagulation reaction may be of sufficient size to allow removal. The flocculation reaction not only increases the size of the floc particles to settle them faster, but also affects the physical nature of the floc, making these particles less gelatinous and thereby easier to dewater.

Thus, representative of typical prior art patents, U.S. Pat. No. 5,328,599,which is incorporated herein by reference is directed to a wastewater treatment system and method for chemical precipitation and removal of metals from wastewater in a continuous or batch treatment process which includes an ion-selective electrode and a reference electrode disposed in a precipitation tank for measuring an electrochemical potential therebetween in a predetermined range. A controller unit is provided which is responsive to the electrochemical potential in the predetermined range and is connected to a precipitant feed unit for automatically controlling the chemical precipitant fed into the precipitation unit.

U.S. Pat. No. 5,645,799 and which is incorporated herein by reference, is directed to an apparatus for optimizing the dosage of a chemical wastewater treatment agent employing a fluorescent tracer. The apparatus includes a series of components that sample the waste stream, process the sample for analysis, analyze the sample, record the data in a historical database, and, based upon the analysis as compared to historical data, adjust the chemical feed system to optimize the chemical wastewater treatment agent according to a programmed optimization logic.

### SUMMARY OF THE INVENTION

It is one aspect of at least one of the present embodiments of the invention to provide for an apparatus and process for monitoring treated water samples prior to discharge to verify that all metals within the discharge have been reduced to specified limits prior to discharge.

It is a further aspect of at least one embodiment of the present invention to provide for an apparatus and process to monitor metal precipitant chemicals in a treated waste water discharge to avoid discharging any such chemicals at levels that could adversely impact a receiving stream or have a detrimental effect on biological activity within a local sewer system.

It is a further aspect of a least one embodiment of the present invention to provide for a process of monitoring a waste water stream which provides a waste water stream sample which is divided into two separate streams. A first stream is directed to a controller that will check for the presence and amounts of metals while the second stream is evaluated for the presence of excess metal precipitants.

It is a further aspect of at least one of the present embodiments to provide for an apparatus and process that upon detection of either one of too much of a metal concentration within a waste stream or too much of a metal precipitant chemical in a waste stream will redirect the waste stream for additional treatment process is prior to discharge.

These and other features, aspects, and advantages of the present invention will become better understood with reference to the following description and appended claims.

### BRIEF DESCRIPTION OF THE DRAWINGS

A fully enabling disclosure of the present invention, including the best mode thereof to one of ordinary skill in the art, is set forth more particularly in the remainder of the specification, including reference to the accompanying drawings.

FIG. 1 is a schematic view of a process and process equipment used for monitoring treated waste water prior to discharge.

### DESCRIPTION OF THE PREFERRED EMBODIMENT

Reference will now be made in detail to the embodiments of the invention, one or more examples of which are set forth below. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used on another embodiment to yield a still further embodiment. Thus, it is intended that the present invention cover such modifications and variations as come within the scope of the appended claims and their equivalents. Other objects, features, and aspects of the present invention are disclosed in the following detailed description. It is to be understood by one of ordinary skill in the art that the present discussion is a description of exemplary embodiments only and is not intended as limiting the broader aspects of the present invention, which broader aspects are embodied in the exemplary constructions.

In describing the various figures herein, the same reference numbers are used throughout to describe the same material, apparatus, or process pathway. To avoid redundancy, detailed descriptions of much of the apparatus once described in relation to a figure is not repeated in the descriptions of subsequent figures, although such apparatus or process is labeled with the same reference numbers.

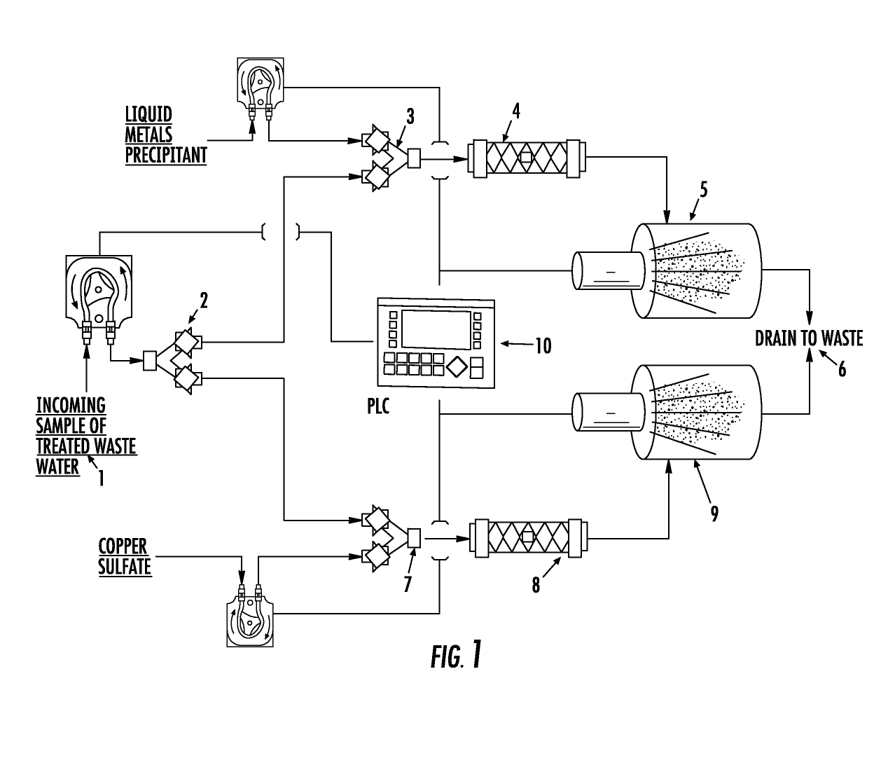

As seen in reference to FIG. 1 at step 1, a sample of treated waste water from a clarifier is introduced into a flow path monitored and regulated by a control unit 10. The control unit 10 is designed to monitor and control testing of the stream flow(s) to make sure all metals have been removed below specified limits and that there are no excess metal precipitant agents or chemicals present in the treated waste water. As seen in numbered step 2, the sample stream is split into two streams with the first stream going to a control system to check for metals and a second stream directed to the portion of the control system that will check for excess metal precipitants or metal precipitating agents. At step 3, the liquid metal precipitant analysis flow is fed into a mixing T which is further mixed and reacted in an inline static mixer at numbered step 4. Any conventional sulfide, carbamate-based, or tri-thio carbonate participating agent may be used as is well known in the art. At step 5, the mixture is directed into a detector where a turbidity meter reads for the presence of turbidity. Metals present in the stream will show up as solids (turbidity) since metals in the solution will precipitate out as metal sulfates or metal carbonates, or metal carbonates. If appropriate discharge limits for metals are met, the mixture will then flow out of the unit and into a waste drain. If the mixture is not ready for discharge, the treated waste water can be further processed to meet compliance standards.

Optionally, the monitoring process may include the use of a digital camera monitoring system which can be utilized to monitor the waste stream. One suitable monitoring system can be seen in reference to Applicant's issued U.S. Pat. No. 8,293,097, issued on Oct. 23, 2012, entitled “System for Continuous Optimization in Waste Water Treatment” and which is incorporated herein by reference. The digital camera based monitoring, system allows an additional level of control and analysis which is useful for some metal containing waste water streams.

At step 7, the second waste stream flow is directed into a second mixing tube where it is mixed with an agent that will precipitate excess metal precipitate agents. One such material is a copper sulfate solution though a number of soluble metal salts and salt solutions may be used. At step 8, the mixture having the copper sulfate or other agent to precipitate excess metal precipitants may be used and flows through a static mixer where it is thoroughly blended and reacted. Step 9, the sample will flow into a second turbidity chamber to detect formation of precipitants which indicates an excess metal precipitant in the stream.

The turbidity readings at step 5 and step 9 are constantly monitored via sacrificial samples so as to provide real time feed back on chemical feed and process conditions of the treated waste water. Upon further treatment, the treated waste water can again be sampled to make certain it complies with discharge standards.

The present disclosure provides a process for monitoring a waste stream that has been treated for removal of metals comprising the steps of:

providing a supply of a treated waste stream, treatment consisting of metal removal steps: taking a first sample of the treated waste stream; taking a second sample of the treated waste stream; mixing the first, sample with a chemical to precipitate metals, the mixing step including use of a static mixer; analyzing the mixed first sample in a turbidity meter to determine whether precipitated metals of present and mixed first sample; mixing the second sample in a static mixer with a precipitating solution to precipitate metal precipitating agents present in the second stream; analyzing the mixed second sample in a turbidity meter to determine whether metal precipitating agents are present in the mixed second sample; discharging the treated waste stream upon verification that the waste stream meets desired levels of both a metal content and a metal precipitating agent content.

The first and second samples, following analysis, may be either discharged with the waste stream or sent for further processing if needed.

Although preferred embodiments of the invention have been described using specific terms, devices, and methods, such description is for illustrative purposes only. The words used are words of description rather than of limitation. It is to be understood that changes and variations may be made by those of ordinary skill in the art without departing from the spirit or the scope of the present invention. In addition, it should be understood that aspects of the various embodiments might be interchanged, both in whole, and in part. Therefore, the spirit and scope of the invention should not be limited to the claims description or the preferred versions contained therein.

That which is claimed:

1. A process for monitoring a waste stream that has been treated for removal of metals comprising the steps of:
providing a supply of a treated waste stream, treatment consisting of metal removal steps:
taking a first sample of the treated waste stream;
taking a second sample of the treated waste stream;
mixing the first sample with a chemical to precipitate metals, the mixing step including use of a static mixer;
analyzing the mixed first sample in a turbidity meter to determine whether precipitated metals of present and mixed first sample;
mixing the second sample in a static mixer with a precipitating solution to precipitate metal precipitating agents present in the second stream;
analyzing the mixed second sample in a turbidity meter to determine whether metal precipitating agents are present in the mixed second sample;
discharging the treated waste stream upon verification that the waste stream meets desired levels of both a metal content and a metal precipitating agent content.
